(19) United States
(12) Patent Application Publication
(10) Pub. No.: US 2024/0161883 A1
(43) Pub. Date: May 16, 2024

(54) HEALTHCARE DATA ACCESS SYSTEM FOR IMPROVING HEALTHCARE DATA USABILITY FOR CLINICIANS AND PATIENTS

(71) Applicants: Steven Douglas Delaney, Markham (CA); Douglas Gordon Schmidt, Markham (CA)

(72) Inventors: Steven Douglas Delaney, Markham (CA); Douglas Gordon Schmidt, Markham (CA)

(21) Appl. No.: 17/986,746

(22) Filed: Nov. 14, 2022

(51) Int. Cl. G16H 10/60

(52) U.S. Cl. CPC G16H 10/60

(57) ABSTRACT

A healthcare data access system for improving healthcare data usability for clinicians and patients comprises at least one computer readable memory device comprising healthcare data of the patients and at least one processor. The processor receives privacy decisions to be applied to the healthcare data. The processor receives a request for the healthcare data from a clinician comprising a selection of a schema. The processor retrieves a set of selected healthcare data from the healthcare data depending on the selected schema and communicates a permissible healthcare data by filtering the selected healthcare data by applying the privacy decisions, or alternatively communicates the set of selected healthcare data from the healthcare data of the patient, by overriding the privacy decisions, when an emergency is declared by the second computing device.

### BACKGROUND

### Field of Invention

The disclosed subject matter relates to the field of healthcare. More particularly, but not exclusively, the subject matter relates to improving healthcare data usability for clinicians and patients.

### Discussion of Prior Art

Healthcare system in many countries is in a period of rapid change fuelled by new technologies, data volume growth, demand for improved services, and a push toward patient-centric healthcare to allow patients greater involvement in their own health. This is straining the healthcare system which is already beset numerous challenges.

Challenges include patient data spread across multiple heterogeneous electronic health record (EHR) systems, making it difficult to share patient data. This makes it difficult for clinicians to obtain a complete patient profile, thereby reducing their efficiency in treating patients. Clinician efficiency is also affected by data quality issues, and poor data presentation.

The implementation of digital health services and patient-centric healthcare is made difficult by the prevalence of older technologies and bureaucratic barriers. The implementation of data privacy and security regulations is similarly challenged by these same issues.

Reducing the strain on the healthcare system requires innovation through technology, as well as overcoming the challenges aggravating the adoption of new technologies. Key to this is improving the usability of patient data in terms of accuracy, privacy, security, presentation, and the sharing of data to provide a complete patient profile. Data usability needs to be improved to support patient-centric healthcare and the efficiency of clinicians who use the data to provide healthcare.

Existing EHR systems are due for a major overhaul. There is documented frustration with user interfaces, interoperability constraints, usability issues and poor IQ. EHR systems must transform to become less transaction oriented and more supportive of clinicians to improve healthcare outcomes. Logic is required in the EHR, such that the clinician is not just presented with data but potential patterns and actions, which are tailored to the healthcare scenario and clinician preferred scenarios.

Further, the data privacy process requires patient approval to share the patient's data between the clinician and the source of the data, such as, another clinician or laboratory. The process is often fraught with delays due to communication lag and the efforts of all the parties involved. Often, clinicians find it easier to simply repeat the tests containing the desired data.

The efficiency of obtaining patient data for the healthcare provider affects the data usability of patient data for the clinician. For this reason, and to improve the data usability for patients to manage their privacy, we selected improving the privacy process as an objective.

### SUMMARY

A healthcare data access system for improving healthcare data usability for clinicians and patients is disclosed. The healthcare data access system may comprise of at least one computer readable memory device comprising healthcare data of the patients and at least one processor. The processor may be configured to receive from a first computing device of a patient, privacy decisions to be applied to the patient's healthcare data. The processor may also receive from a second computing device of a clinician a request for the healthcare data of the patient. The request may comprise a selection of a schema. The schema may be selected from a plurality of schemas. Each of the schemas may correspond to a pre-defined search query. Further, the processor may be configured to retrieve a set of selected healthcare data from the healthcare data of the patient, based on the request comprising the selected schema and communicate a permissible healthcare data to the second computing device. The permissible healthcare data from the selected healthcare data may be obtained by filtering the selected healthcare data by applying the privacy decisions received from the first computing device or communicate the set of selected healthcare data from the healthcare data of the patient, by overriding the privacy decisions received from the first computing device, when an emergency is declared by the second computing device.

### BRIEF DESCRIPTION OF DRAWINGS

Embodiments are illustrated by way of example and not limitation in the figures of the accompanying drawings, in which like references indicate similar elements and in which:

FIG. 1 illustrates a healthcare data access system 100, in accordance with an embodiment.

FIG. 2A depicts a logical architecture of a Patient Consent System (PCS) front end module 104, in accordance with an embodiment.

FIG. 2B depicts a logical architecture of a PCS back end module 106, in accordance with an embodiment.

FIG. 3 illustrates relationships defined between plurality of nodes, in accordance with an embodiment.

FIG. 4 illustrates a graph DB 108 model design, in accordance with an embodiment.

FIG. 5 depicts a segment of the graph DB 108 to illustrate data retrieval process, in accordance with an embodiment.

FIG. 6 shows a segment of the graph DB 108 to illustrate pattern matching process, in accordance with an embodiment.

FIG. 7 depicts a segment of the graph DB 108 to illustrate data generation process, in accordance with an embodiment.

FIG. 8 depicts the detailed view of a Healthcare Discovery Patient Record Plus (HDPR+) front end module 112 and a HDPR+ back end module 110, in accordance with an embodiment.

FIG. 9 illustrates interaction of a patient 102 on a computing device corresponding to the patient 102, in accordance with an embodiment.

FIG. 10 illustrates interaction of a clinician 114 on a computing device corresponding to the clinician 114, in accordance with an embodiment.

FIG. 11A-11D illustrate a flowchart 1100 depicting the workflow of the patient 102 using a PCS 200 within the system 100, in accordance with an embodiment.

FIG. 12A-12B illustrate a flowchart 1200 depicting the workflow of the clinician 114 using a HDPR+ system 800, in accordance with an embodiment.

FIG. 13 illustrates a computing device 1300, in accordance with an embodiment.

### DETAILED DESCRIPTION

The following detailed description includes references to the accompanying drawings, which form a part of the detailed description. The drawings show illustrations in accordance with example embodiments. These example embodiments, which may be herein also referred to as “examples” are described in enough detail to enable those skilled in the art to practice the present subject matter. However, it may be apparent to one with ordinary skill in the art, that the present invention may be practised without these specific details. In other instances, well-known methods, procedures and components have not been described in detail so as not to unnecessarily obscure aspects of the embodiments. The embodiments can be combined, other embodiments can be utilized, or structural, logical, and design changes can be made without departing from the scope of the claims. The following detailed description is, therefore, not to be taken in a limiting sense, and the scope is defined by the appended claims and their equivalents.

In this document, the terms “a” or “an” are used, as is common in patent documents, to include one or more than one. In this document, the term “or” is used to refer to a nonexclusive “or,” such that “A or B” includes “A but not B,” “B but not A,” and “A and B,” unless otherwise indicated.

### Healthcare Data Access System

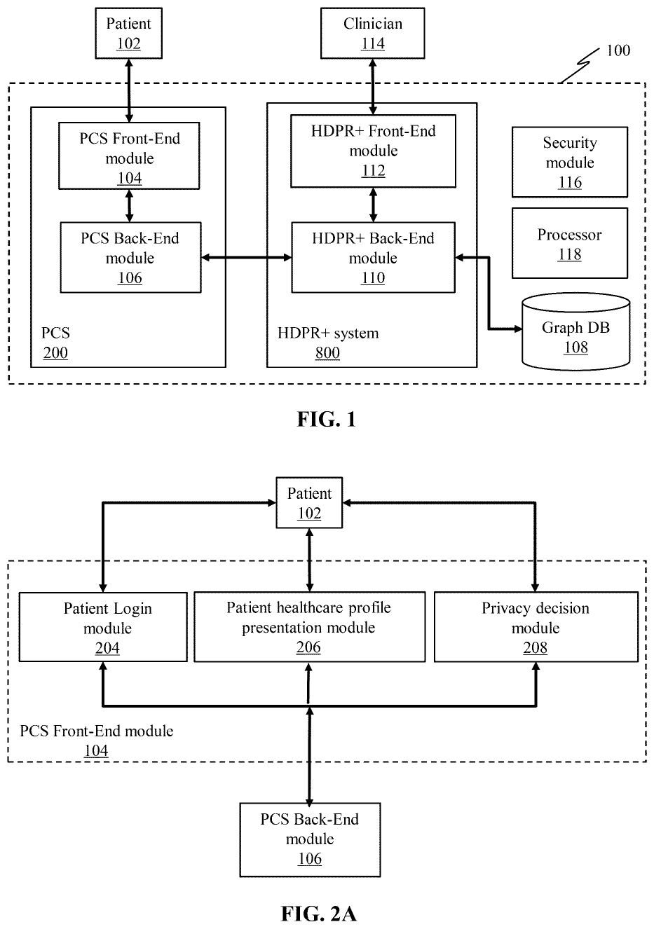

FIG. 1 illustrates a healthcare data access system 100, in accordance with an embodiment. The healthcare data access system 100 includes a patient 102, a healthcare provider 114, a patient consent system (PCS) 200, graph database (graph DB) 108, a healthcare discovery patient record plus (HDPR+) system 800 and a security module 116. The healthcare provider 114 may be, but not limited to, a clinician or a first responder.

In an embodiment, the PCS 200 may comprise of a PCS front end module 104 and a PCS back-end module 106.

In an embodiment, the HDPR+ system 800 may comprise of HDPR+ front end module 112 and a HDPR+ back-end module 110.

### Patient Consent System

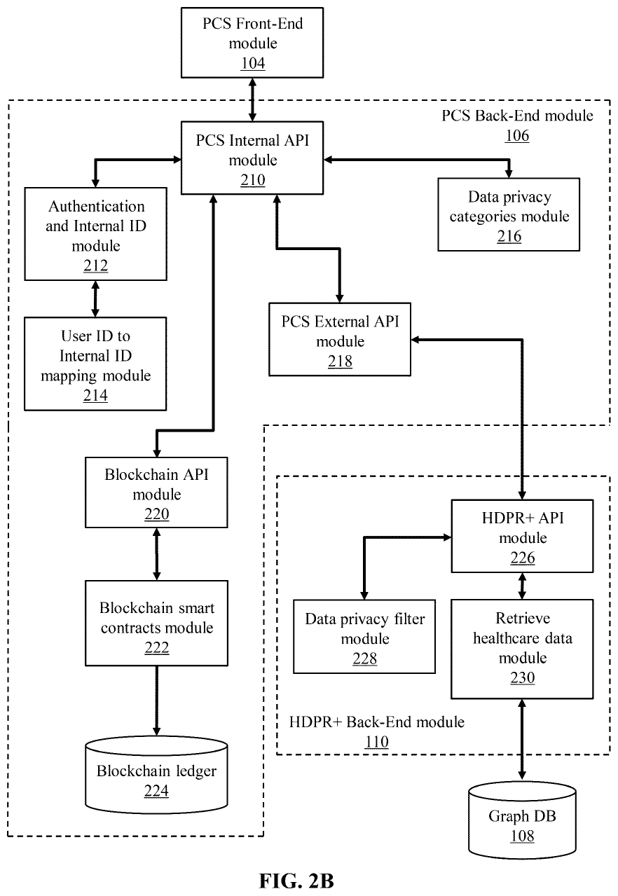

FIG. 2A depicts the logical architecture of the PCS front end module 104, in accordance with an embodiment.

In an embodiment, the PCS front end module 104 includes a patient user interface (UI), wherein the patient UI comprises of a patient login module 204, a patient healthcare profile presentation module 206 and a privacy decision module 208. The patient login module 204 may enable the patient 102 to login to the system 100 through multi-level authentication. The patient healthcare profile presentation module 206 may enable the patient 102 to view their healthcare profile. The privacy decision module 208 may allow the patient 102 to view/set/edit privacy decisions against privacy categories and/or time periods i.e; allow the patient 102 to accurately select the data to flag as private by the data category and/or a date range. The patient UI may provide the ability to authorize specific data flagged as private to be viewed by a specific clinician who has requested access. Further, the patient UI may allow the patient 102 to record and edit their privacy decisions.

FIG. 2B depicts the logical architecture of the PCS back end module 106, in accordance with an embodiment.

In an embodiment, the PCS back end module 106 may comprise of a PCS internal application programming interface (API) module 210, an authentication and internal ID module 212, a user ID to internal ID mapping module 214, a data privacy categories module 216, a PCS external API module 218, a blockchain API module 220, a blockchain smart contracts module 222, and a blockchain ledger 224.

In an embodiment, the PCS internal API module 210 may allow the PCS front end module 104 and the PCS back end module 106 to securely communicate with each other. The PCS internal API module 210 may be configured to handle requests from the PCS external API module 218 such as, but not limited to, the HDPR+ back end module 110. The HDPR+ back end module 110 may request a copy of a patient's 102 privacy decisions which may enable the HDPR+ back end module 110 to filter private data of the patient 102 before presenting to a viewer, such as the clinician 114. All activities of the PCS internal API module 210 and the PCS external API module 218 may be logged into the blockchain ledger 224 by the blockchain API module 220.

In an embodiment, each patient 102 (herein after may also be referred to as a user) may be assigned an internal ID used to index all data on the blockchain ledger 224 belonging to that user 102. Data on the blockchain ledger 224 belonging to a particular user 102 may include their privacy decisions and log entries related to their healthcare data. The user authentication and internal ID module 212 may allow the user 102 to login and map the user 102 to their respective internal ID via the user ID to internal ID mapping module 214. The user 102 may never know the value of their internal ID. This may add a layer of security and may further allow the user 102 to access their data on the blockchain ledger 224 by remapping their internal ID. The authentication and internal ID module 212 may also be used by the PCS external API module 218 to retrieve the patient's 102 healthcare decisions.

In an embodiment, the data privacy categories module 216 may identify the type of data from the perspective of privacy. As an example, categories may include, but not limited to, surgery, psychiatric, sexual, drugs and diseases. Categories and dates may be assigned as metadata to the patient's healthcare data provided by the HDPR+ back end module 110. Categorizing data may make it easier for the patient 102 to identify which data should be made private without requiring strong knowledge of medical terminology.

In an embodiment, the patient 102 may be allowed to choose to flag their healthcare data as private using time periods unrelated to the categorization used. As an example, patient 102 may flag all data categorized “psychiatric” as private, or data flagged as “sexual” in the date range from Jan. 10, 2003 to Mar. 15, 2004. Another option may allow the patient 102 to flag all healthcare data as private within a date range.

In an embodiment, the blockchain API module 220 may be connected to a blockchain ledger 224 (herein after may also be referred as a blockchain database (blockchain DB)). The blockchain DB 224 may use, but not limited to, Hyperledger fabric, Ethereum and/or Parity as the blockchain technology.

In an embodiment, the blockchain DB 224 may hold all patient privacy decisions and activity log entries. All the blockchain transactions may be indexed by the user's internal ID. Further, the blockchain transactions may be saved, read, encrypted and unencrypted by a PCS back end user ID. Patients 102 may request the PCS back end module 106 via the PCS internal API module 210 to save and retrieve their privacy decisions and healthcare data for viewing.

In an embodiment, the PCS external API 218 may be configured to authenticate external systems and retrieve the patient's privacy decisions from the blockchain DB 224.

In an embodiment, the PCS external API module 218 may be configured to authenticate the external systems before allowing access. The PCS external API module 218 may further retrieve the privacy decisions of the patient 102, which may prevent external users of the external system from viewing the patient data flagged as private. External system (such as the HDPR+ system 800) may provide information such as, the name of the clinician 114 who is requesting access to the patient's 102 data.

In an embodiment, depending on the regulatory requirements of the jurisdiction, clinicians 114 may have the right to request viewing all of the patient's 102 healthcare data if they declare a medical emergency involving the patient 102. During which, the external system may be configured to ignore the patient's privacy decisions received from the PCS external API module 218. Further, the name of the clinician 114 and their decision of declaration of emergency may be stored in the blockchain DB 224.

In an embodiment, the HDPR+ back end module 110 may comprise of a HDPR+ API module 226, a data privacy filter module 228, and a retrieve healthcare data module 230.

In an embodiment, the HDPR+ API module 226 may be configured to request the retrieval of the patient's 102 privacy decisions from the PCS back end module 106. Further, the HDPR+ API module 226 may respond to requests from the PCS external API module 218 and other external systems to retrieve the patient's 102 healthcare profile. The PCS internal API module 210 may then present this data to the patient 102 to help them determine which healthcare data should be flagged as private.

In an embodiment, the retrieve healthcare data module 230 may retrieve data from the graph DB 108 when requested by the clinician 114.

In an embodiment, the data privacy filter module 228 may be configured to filter the data retrieved by the HDPR+ API module 226 before presenting it to the requesting clinician 114.

### Graph Database (Graph DB)

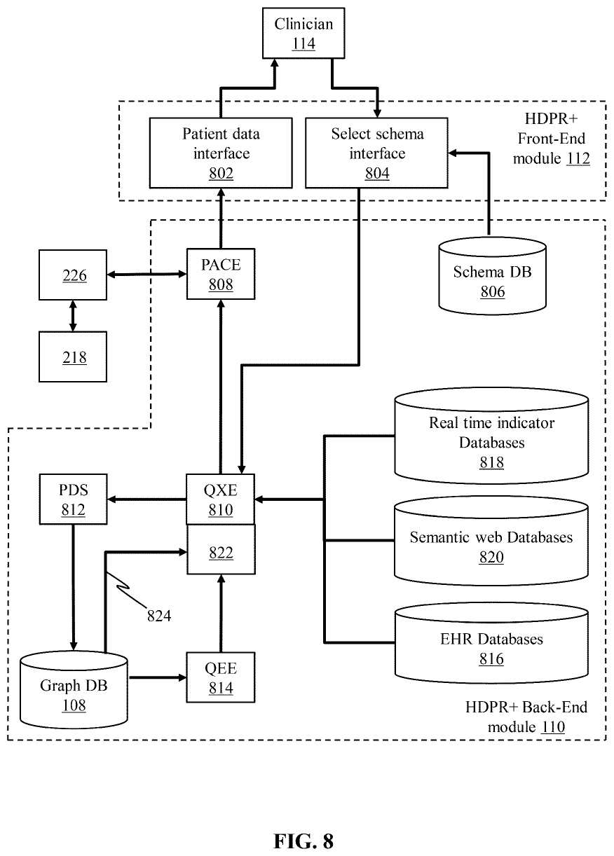

In an embodiment, the graph DB 108 residing on a computer readable memory device, may be configured to host the patient's healthcare profile and associated data which may include, but not limited to, the privacy classification of the patient's healthcare data. The graph DB 108 may further be configured to pull and store data from an electronic health records database (EHRDB) 816 and a laboratory DB (please refer FIG. 8).

In an embodiment, the graph DB 108 may be based on Neo4j technology. The graph DB 108 may be configured to store patient data in presentation data standard (PDS) format with relationships that support easy retrieval of patient data, metadata and medical references on patient data, algorithms for creating generated data, support for pattern matching and support for Schema data selection. The graph DB 108 may be a temporary repository, whereas the EHRDB's 816 and other databases may be considered as the permanent repositories of the patient data.

In an embodiment, the graph DB 108 of the proposed system 100 may be configured to delete unused nodes and their relationships after a set period of time.

In some embodiment, the medical references may be Uniform Resource Locator (URL) reference or semantic web query to medical information such as, drug dosage information.

In an embodiment, the graph DB 108 may be configured to delete the data that is not accessed for a set period to ensure better performance.

In an embodiment, the graph DB 108 may be configured to support plurality of data types required by a broad range of clinicians 114. The plurality of data types may include information regarding, but not limited to, demographics, drug usage, medical history (diseases such as dermatological, non-dermatological, addiction etc.), family history (addiction, non-addiction, dermatological, etc), surgical, psychiatric history, vital signs, and lifestyle.

In an embodiment, the graph DB 108 may be configured to include pattern matching. The key function of pattern matching is to test for medical conditions through a positive pattern match. The medical conditions to be tested may be requested by the Schema. Pattern matching is discussed in greater detail later in the description.

In an embodiment, the graph DB 108 may be configured to process the patient data based on an algorithm to process the data. The algorithm may be a request to provide a matrix of data suitable for presentation as a chart or graph. For example, a matrix representing a patient's 102 weight over time. This data is more useful to the clinician 114 if presented as a graph.

In an embodiment, the graph DB 108 may be configured to store medical information which may refer to a URL or Universal Resource Identifier (URI) reference to information such as, drug dosage and side effects, disease symptoms, and drugs commonly used for specific surgeries. This type of information may be helpful to the clinician 114 or intern in training.

In an embodiment, plurality of nodes may be created to hold patient data at a granular level, for example, blood type. The graph DB 108 may be configured to store the plurality of nodes. The graph DB 108 may be configured to create simple relationship groupings at the granular level and continue to combine relationship groupings to create more complex relationships. Further, the graph DB 108 may be configured to decompose complex groupings into smaller comprehensible relationship groupings that are more easily understood, managed and adjusted. Additionally, the graph DB 108 may be configured to nullify older relationships.

In an embodiment, the stored plurality of nodes may be named and stored as, but not limited to, a Schema node (Sc), a Patient node (P), a PatientData node (PD), an Entity node (E), a NullEntity node (NE), a Supplement node (Su), a Condition node (C) and a Presentation node (Pr).

In an embodiment, the Schema node (Sc) may represent a healthcare scenario and/or a clinician speciality. This descriptive information may be stored in the Schema node (Sc) and may be displayed to help the clinician 114 select the Schema that best meets their needs. The Schema node (Sc) may contain relationships to the nodes identifying the patient data to be retrieved and the medical conditions to be tested. The Schema node (Sc) may further be personally customized allowing the clinician 114 to identify additional data and medical condition checks. The Schema node (Sc) may also carry information with instructions on how the data should be presented. The presentation may be optimized for the healthcare scenario represented by the Schema. Further, the Schema node (Sc) may be attached to other Schema nodes (Sc) so that the data most important is displayed first and quickly. Moreover, additional data may be retrieved in the background and made ready for viewing when the retrieved data is complete. This would help first responders 114. A first responder 114 may get basic information on a patient 102 without having to wait until all data has been retrieved.

In an embodiment, the Patient node (P) may be assigned to each of the registered patients 102. The Patient node (P) may contain information a query execution engine uses to know how to retrieve patient data from the EHRDB's 816 and laboratory databases. The Patient node (P) may act as a vertex for access to all the patient's 102 data, wherein vertex may be an indexed node. Indexed node may allow faster access to a vertex node, thereby improving the performance of a query

In an embodiment, the PatientData node (PD) may contain a single attribute of patient data retrieved from an EHRDB 816 or another database. Further, the PatientData node (PD) may be related to an Entity node (E) that contains metadata about the type of healthcare data contained in the PatientData node (PD).

In an embodiment, the Entity node (E) may be created for each attribute of the healthcare data. For example, there will be an Entity node (E) for the patient's age, blood oxygenation level or a drug. The Entity nodes (E) may contain metadata on the type of patient data. The privacy category value of the Entity node (E) allows the patient 102 to easily make private, all patient data related to an Entity node (E) with the same category value. The Entity nodes (E) may be configured to join PatientData nodes (PD) to the Schema nodes (Sc), the Condition nodes (C), the Supplementary nodes (Su), and the Presentation nodes (Pr).

In an embodiment, any PatientData node (PD) that does not have an associated Entity node (E) may be related to the NullEntity node (NE). This may ensure that all patient data is retrieved and not ignored. This may indicate that the Entity node (E) definition is probably missing.

In an embodiment, the Supplement node (Su) may contain an URL or semantic web query to retrieve medical information from external sources. As an example, the Entity node (E) for a specific drug may be related to the Supplement node (Su) with a reference to information on that drug.

In an embodiment, the Condition nodes (C) may contain algorithms that will test (pattern match) for the medical condition the node represents. The algorithm variables may be pulled from PatientData nodes (PD) through relationships from the Condition node (C) to one or more Entity nodes (E). Examples of conditions may include, but not limited to, high blood pressure, alcoholism and drug combination side effects.

In an embodiment, the Presentation nodes (Pr) may indicate which patient data will be presented as defined by the Schema linked to this node. The presentation node (Pr) may contain instructions on how the data should be presented on the screen using variables saved in the relationships to each Entity. The Presentation nodes (Pr) may further be configured to include instructions to generate data such as a matrix of data to be presented as a graph to the viewer. Common groups of data such as, demographic data on the patient, may be defined in one Presentation node (Pr) rather than being defined repeatedly for each Schema node (Sc).

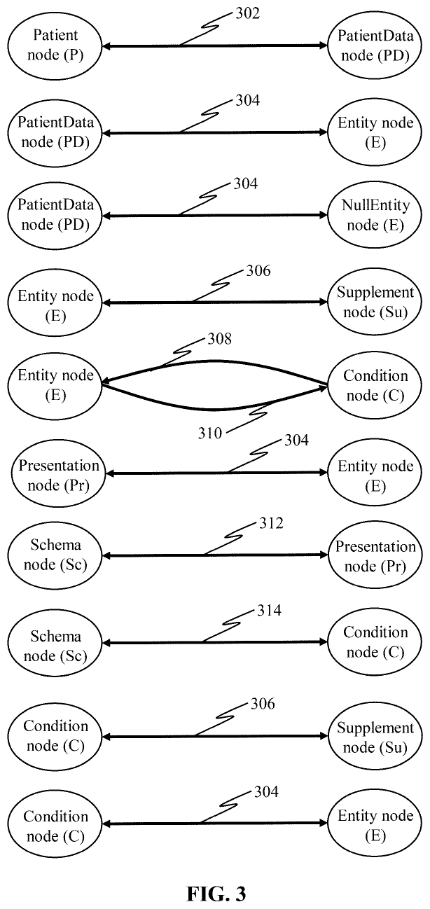

In an embodiment, the graph DB 108 may be configured to store plurality of relationships. The plurality of relationships may be defined as, but not limited to, a PATIENT_INFO relationship 302, an ENTITY_REF relationship 304, a SUPP_REF relationship 306, a GUAGE relationship 308, an INDICATOR relationship 310, a PRESENT relationship 312, and a TEST relationship 314. FIG. 3 illustrates the relationships defined between the plurality of nodes, in accordance with an embodiment.

In an embodiment, the PATIENT_INFO relationship 302 may connect a single Patient node (P) to multiple PatientData nodes (PD) (each of which holds a single piece of patient data). The PATIENT_INFO relationship 302 may be used in queries to identify all the healthcare data belonging to a specific patient 102.

In an embodiment, the ENTITY_REF relationship 304 may connect the PatientData node (PD) to the Entity node (E) which contains metadata specific to the type of data stored inside the PatientData node (PD). The ENTITY_REF relationship 304 may be used in queries to identify specific types of patient data and to obtain information (metadata) on the patient data. The Entity node (E) may represent a single piece of healthcare data. For example, a person's weight. Every patient who has data containing their weight will have a relationship to the same Entity node (E) that represents weight. In case wherein the patient has multiple weights because their weight was recorded once each year then, that single patient will have multiple PatientData nodes (PD), all pointing to the same Entity node (E). The use of multiple relationships to the same Entity node (E) keeps the queries simple and, in cases wherein the patient 102 has multiple weights, it allows the query to easily retrieve multiple weights each with a different date. This in turn may be easily provided to a presentation service that would chart the patient's 102 weight over time. This information may be of greater value to the clinician 114, than the display of a single weight value.

In an embodiment, the ENTITY_REF relationship 304 may connect the PatientData node (PD) to the NullEntity node (NE). This relationship may be used only if the type of patient data retrieved cannot be identified and therefore, the system does not know which Entity node (E) to connect the data to. There may be only one NullEntity node (NE). The ENTITY_REF relationship 304 to the NullEntity node (NE) may be used in queries to detect patient data that cannot be displayed. It alerts the tech teams to determine why this data does not have a corresponding Entity node (E).

In an embodiment, the SUPP_REF relationship 306 may be configured to connect the Entity node (E) to external references containing medical information on the type of data the Entity represents. The SUPP_REF relationship 306 may be used in queries to allow the clinician to link to medical information concerning the patient data. For example, an Entity node (E) representing a specific drug would have a SUPP_REF relationship 306 to a Supplementary node (Su) providing information on the drug.

In an embodiment, the GUAGE relationship 308 may connect the Condition node (C) to the Entity node (E). Queries checking for pattern matches would use the GUAGE relationship 308 to identify the patient data values to be plugged into the algorithm that determine whether there is a matching pattern.

In an embodiment, the INDICATOR relationship 310 connects the Entity node (E) to the Condition node (C) for queries which may want to determine which specific piece of patient data is used to determine the medical conditions.

In an embodiment, the ENTITY_REF relationship 304 may connect the Presentation node (Pr) to the Entity node (E). This relationship may be used in queries to identify a group of several Entities for one of three purposes. First, the ENTITY_REF relationship 304 may be used to retrieve common pieces of patient data that are often displayed together. This reduces the number of relationship connections in the database and the number of data Entities contained in the query. Second, the Presentation node (Pr) may be used to generate new data. An algorithm in the Presentation node (Pr) may collect patient data through the Entities to compute new data. For example, the Presentation node (Pr) may retrieve a history of blood pressure values to create the information needed for the data to be displayed as a line graph to show and project changes over time. Third, the Presentation node (Pr) may contain instructions controlling the way data is presented. For example, instructions for determining where on the screen, data is to be displayed.

In an embodiment, the PRESENT relationship 312 may connect the Schema node (Sc) to one or more Presentation nodes (Pr) to indicate which patient data may be displayed and how it may be presented. The PRESENT relationship 312 may be used in a query to simplify the identification of which patient data may be retrieved by the Schema. The PRESENT node 312 may also be used to generate new patient data based on a clinical algorithm.

In an embodiment, the ENTITY_REF relationship 304 may connect the Schema node (Sc) directly to one or more Entity nodes (E) to identify the data to be retrieved. This is an alternative for use of the Presentation node (Pr) to identify the data to be retrieved. Here, the presentation of the data is identified in the Entity node (E) and control over placement of the data will be limited.

In an embodiment, the TEST relationship 314 may connect the Schema node (Sc) to a Condition node (C) containing an algorithm to be used to determine if the medical condition represented by the Condition node (C) is present or not. The TEST relationship 314 simplifies the query by removing the need to specify which medical conditions are to be tested by the Schema.

In an embodiment, the SUPP_REF relationship 306 may connect a Condition node (C) to external references containing medical information on the medical condition represented by the Condition node (C). The SUPP_REF relationship 306 may be used in queries to allow the clinician to link to medical information concerning the medical condition.

In an embodiment, the ENTITY_REF relationship 304 may connect a Condition node (C) to the Entity nodes (E) representing the patient data values for the pattern matching algorithm. The Entity nodes (E) identified by the Condition node (C) identify in turn, the patient data to be retrieved for the algorithm.

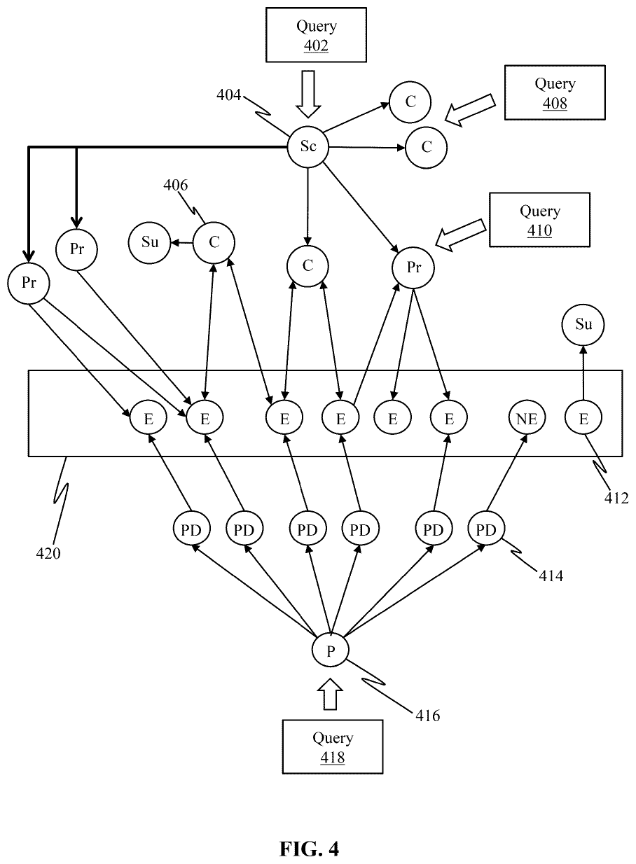

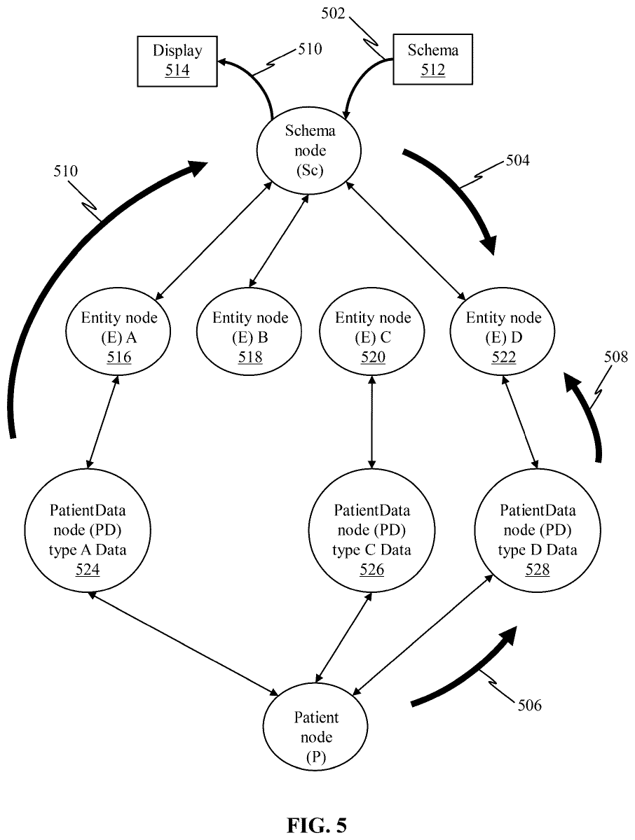

FIG. 4 illustrates the graph DB 108 model design, in accordance with an embodiment. The design of the graph DB 108 may be founded on the intersection of the Entity nodes (E) of a query from the top-down and/or with a query from the bottom-up.

In an embodiment, a top-down query may start with the Schema node (Sc) and a bottom-up query may start with the Patient node (P). The Entity nodes (E) which have a path of links to both the Schema node (Sc) and the Patient node (P) may represent the list of intersecting Entity node (E). These Entity nodes (E) may represent the patient data most important to the clinician 114 for the healthcare scenario represented by the Schema.

Referring to FIG. 4, according to an embodiment, queries 402 using the Schema node (Sc) 404 may be the starting vertex, which may test more than one medical condition.

In an embodiment, referring to FIG. 4, a Condition node (C) 406 may stand alone i.e; the Condition node (C) may be queried without having to use a Schema node (Sc). As an example, the Condition node (C) 406 may be used to test the side effects of mixing two drugs, wherein each drug is represented by an Entity node (E).

In an embodiment, queries 408 using the Condition node (C) may be the starting vertex. Alternatively, queries 410 using the Presentation node (Pr) may be the starting vertex. An Entity node (E) 412 that may relate to a PatientData node (PD) if the patient 102 has data related to that Entity. PatientData node (PD) 414 may contain electronic health record (EHR) patient data. A patient 102 may have a Patient node (P) 416, but no PatientData node (PD) until the patient's 102 EHR data is queried. Further, queries 418 using the Patient node (P) 416 may be the starting vertex. Entity nodes (E) 420 may form the intersection between the Patient vertex queries and the Schema vertex queries. This may allow easy identification of patient data to be used for pattern matching algorithms and identify supplementary data for the patient data.

In an embodiment, there may be three basic graph DB 108 query algorithms upon which all other queries may be based, which are: Data Retrieval, Pattern Matching and Data Generation.

In an embodiment, Data Retrieval may identify the patient data to be retrieved and the importance of the data to the clinician 114.

In an embodiment, Pattern Matching may match patterns to determine whether the patient 102 might be suffering from a specified medical condition.

In an embodiment, Data Generation may describe how new patient data may be generated for viewing.

FIG. 5 depicts a segment of the graph DB 108 to illustrate the data retrieval process, in accordance with an embodiment. Referring to FIG. 5, steps 502 and 504 may represent a top-down query. At step 502, the clinician 114 may select a Schema 512 to retrieve patient data for viewing through the Schema node (Sc). At step 504, the graph DB 108 identifies the type of patient data the clinician 114 may want to view, wherein the Schema node (Sc) is related to the Entity node (E) A (516), the Entity node (E) B (518), and the Entity node (E) D (522).

Referring to FIG. 5, steps 506 and 508 may represent a bottom-up query. At step 506, all the related PatientData nodes (PD) type A, C, D (524, 526, 528), and the Entity nodes (E) A, C, D (516, 520, 522) may be identified for the patient specified by the clinician 114.

At step 508, the intersection of the Entity nodes (E) A, B, D (516, 518, 522) and the Entity nodes (E) A, C, D (516, 520, 522) may be identified, which is the Entity node (E) A (516) and the Entity node (E) D (522) in this case.

Referring to FIG. 5, step 510 may represent the data retrieved from the PatientData nodes (PD). At step 510, on the display screen of the clinician 114, patient data from the PatientData nodes (PD) type A data (524) and the PatientData nodes (PD) type D data (528) may be displayed on a display 514 along with a notification that data from the PatientData nodes (PD) type B data is unavailable.

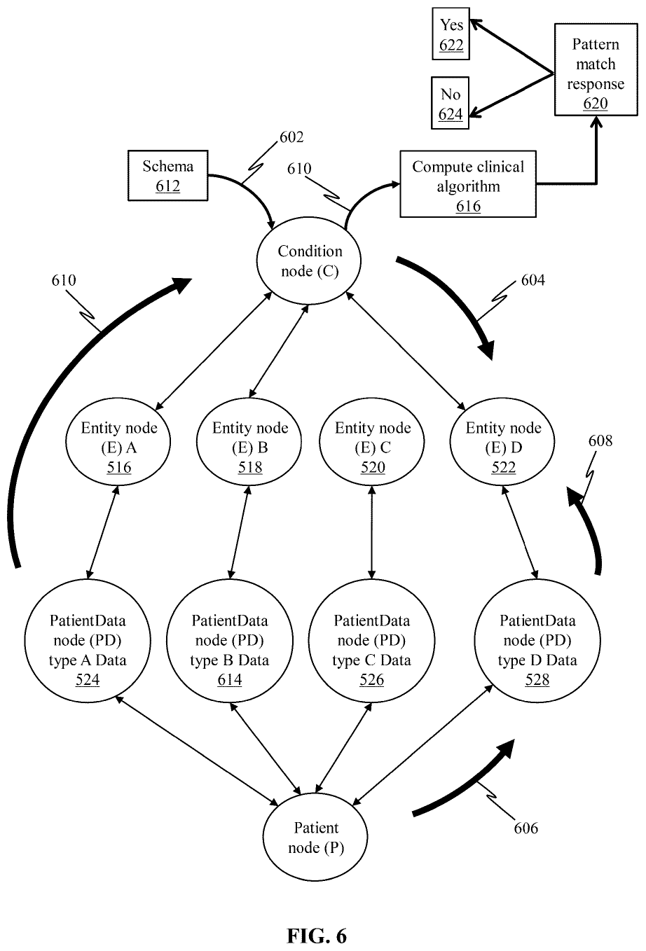

FIG. 6 shows a segment of the graph DB 108 to illustrate the pattern matching process, in accordance with an embodiment. Referring to FIG. 6, at step 602, a Schema 612 may initiate a check for a medical condition represented by a Condition node (C).

At steps 604, 606 and 608, a data retrieval algorithm may retrieve data from the PatientData node (PD) type A data (524), the PatientData node (PD) type B data (614) and the PatientData node (PD) type D data (528).

At step 610, the data from the PatientData node (PD) type A data (524), the PatientData node (PD) type B data (614) and the PatientData node (PD) type D data (528) may be sent back to the Condition node (C), which may compute a clinical algorithm 616 designed to determine if the patient's data indicates a positive response to the medical condition.

At step 618, the graph DB 108 verifies the pattern match response 620. At step 622, if the pattern match response 620 is a Yes, it is the responsibility of the clinician 114 to confirm the medical condition and ensure that proper healthcare is provided to the patient 102.

At step 624, if the pattern match response 620 is a No, then the clinical Schema 612 cannot be executed, and notification is sent to the clinician that not all the data was available to perform the test.

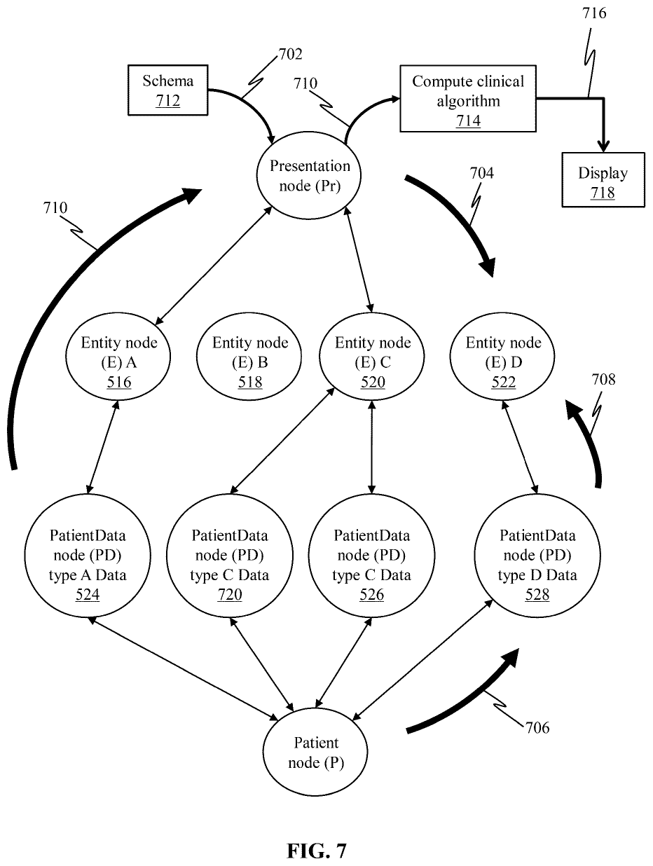

FIG. 7 depicts a segment of the graph DB 108 to illustrate the data generation process, in accordance with an embodiment. Referring to FIG. 7, at step 702, a Schema 712 initiates a Presentation node (Pr) to retrieve patient data. In an embodiment, the Schema 712 may retrieve data without processing. In another embodiment, the Schema 712 may retrieve data to generate new data.

At steps 704, 706 and 708, the data retrieval algorithm may retrieve data from the PatientData node (PD) type A data (524), the PatientData node (PD) type C data (720), and the PatientData node (PD) type C data (526). In an embodiment, the PatientData node (PD) type C data (720) and the PatientData node (PD) type C data (526) may represent the same type of patient data. The difference between the PatientData node (PD) type C data (720) and the PatientData node (PD) type C data (526) may be the date the data was collected, for example, body weight.

At step 710, the PatientData node (PD) type A data (524), the PatientData node (PD) type C data (720), and the PatientData node (PD) type C data (526) may be sent to the Presentation node (Pr) to execute an algorithm 714 to generate data.

At step 716, the patient data from the PatientData node (PD) type A data (524) may be sent to display 718, and an array created with the data from the PatientData node (PD) type C data (720), and the PatientData node (PD) type C data (526) may be used by the Presentation node (Pr) to generate a graph of the patient weight over time.

In an embodiment, the data privacy filter module 228 may be configured to filter out the data flagged as private by the patient 102 to the clinician 114.

In an embodiment, the system 100 may include a Schema which may be predefined. The predefined Schema may help the clinician 114 to retrieve the patient data most useful to the healthcare scenario and may be based on the speciality of the clinician 114. The schema may include, but not limited to, tests for medical conditions, information to optimize the presentation of the patient's 102 data to the clinician 114.

### HDPR+ System

FIG. 8 depicts the detailed view of the HDPR+ front end module 112 and the HDPR+ back end module 110, in accordance with an embodiment.

In an embodiment, the HDPR+ back end module 110 may include a schema database (schema DB) 806, a query execution engine (QXE) 810, a presentation data standard (PDS) module 812, a query expansion engine (QEE) 814 and data privacy and access control engine (PACE) 808.

In an embodiment, referring to FIG. 8, the HDPR+ front end module 112 may be configured to include a clinician UI. The clinician UI may include a patient data interface 802 and a select schema interface 804.

In an embodiment, the patient data interface 802 may be configured to present patient data to the clinician 114 and allow the clinician 114 to navigate through presented patient data.

In an embodiment, the schema DB 806 may store predefined Schemas. The clinician 114 may be asked to select a schema, retrieved from the schema DB 806. All the patient's 102 data, except data flagged as private, may be available to the clinician 114, with the most important data presented first according to the instructions in the schema. Schema definitions may be governed by a committee of clinicians to ensure that the data retrieved meets the needs of most clinicians and healthcare scenarios. Provision may be made to allow clinicians to create or customize their own Schemas.

In an embodiment, both the Schema node (Sc) and the Presentation node (Pr) may contain instructions on how the data is to be presented.

In an embodiment, the QXE 810 may retrieve all the data found about the patient from the EHRDB's 816 and other sources such as, but not limited to, laboratory DB, a semantic web databases 820, and a real time indicator databases 818. The QXE 810 may further be configured to store the retrieved data in the graph DB 108. The QXE 810 may further be configured to execute the schema selected by the clinician 114 and consolidate the retrieved data.

In an embodiment, the real time indicator databases 818 may be configured to share the patient's 102 real time data received from devices such as, but not limited to, smart watches, and applications on the mobile phone of the patient 102.

In an embodiment, the semantic web databases 820 may store medical information related to the patient's healthcare data.

In an embodiment, the PDS module 812 is configured to transform the data retrieved from plurality of databases such as the EHRDB's 816 and laboratory databases into a common PDS format. The PDS module 812 may store the transformed PDS format data from external sources in the graph DB 108. The transformation of data into PDS format may retain the semantic meaning and value of the retrieved data. The PDS format is meant for use only within the HDPR+ back end module 110, in accordance with an embodiment.

In an embodiment, the QEE 814 may be configured to provide supplemental data to the clinician 114, requesting the patient's 102 data, relevant to the healthcare scenario defined by the schema. Supplemental data may be defined as additional patient data derived from the patient data retrieved from external databases. It may comprise of the results of medical condition tests, generated data and medical information. Further, the QEE 814 may be configured to transmit the data to the QXE 810 for choreograph and consolidation of data 822.

In an embodiment, the graph DB 108 may be configured to send the patient and presentation data 824 to the QXE 810 for choreograph and consolidation of data 822.

In an embodiment, the PACE 808 may be configured to retrieve patient's privacy decisions from the PCS external API module 218 to filter out data the patient has flagged as private. The PACE 808 may further be configured to push remaining data to the patient data interface 802 for display to the clinician 114.

In an embodiment, the PACE 808 may be configured to provide data on the clinician 114 viewing the data as well as the date and time for logging the request into the blockchain DB 224.

In an embodiment, the data privacy filter module 228 may be same as the PACE 808.

In an embodiment, the patient data interface 802 may be configured to position the data on the screen in a manner that allows the clinician 114 to easily view the most useful data for the healthcare scenario and speciality of the clinician 114. Further, the patient data interface 802 may present some data as text or as charts. The QXE 810 may be instructed to present data in a specific format via the consolidation function. Instructions about the data presented may be provided by the Schema and metadata from the graph DB 108.

In an embodiment, software's such as, but not limited to, Google charts and Tableau, may be used to display the data to the clinician 114.

In an embodiment, navigation option may be provided for the data that does not fit on a display device of the clinician 114. The data may be provided in the form of starburst charts, in accordance with an embodiment.

### Security Module

In an embodiment, the security module 116 may be configured to provide data security and privacy protection. The security module 116 may provide data security and privacy protection to the patient healthcare data which may be retrieved from various EHRDB's 816 and laboratory sources. Further, the security module 116 may provide data security and privacy protection to the data related to the patient's privacy decisions recorded on the blockchain DB 224.

In an embodiment, data security may be categorized as data at-rest, data in-use and data in-transit.

In an embodiment, data at-rest may be the data residing in graph DB 108 and the blockchain DB 224. The HDPR+ system 800 may use the graph DB 108 to hold patient data for viewing which may be based on Neo4j technology.

In an embodiment, the security protections put in place may include patient ID's, which may be a unique system generated random number associated with the patient's healthcare registration number. For example, in Ontario, Canada, the patient's healthcare registration number may be their OHIP number assigned to them when registering with the Ontario health system. This association may be stored in a secure and encrypted table within the back-end layer. The back-end will use the internal patient ID to retrieve the patient's data. Neither the patient nor the clinician has access to the generated ID number and may have access to only to the patient's healthcare number.

In an embodiment, for performance reasons and to ensure proper execution of queries, encryption of data in graph DB 108 may be performed on data that may be used to directly identify the patient 102. To mitigate the risk to a certain extent, the graph DB 108 may be configured to delete the data in the graph DB 108 after a period of non-use. Further, the patients ID may use a generated ID which is not directly accessible to any user.

In an embodiment, all the data store in the blockchain DB 224 in the PCS 200 may be encrypted.

In an embodiment, data in-transit may be defined as the patient data that is being processed or actively being viewed by the clinician 114. During this, the data may reside in a computer memory. Further, all data being processed may be kept in the PCS back end module 106 or the HDPR+ back end module 110 while the data being viewed may be kept in the PCS front end module 104 or the HDPR+ front end module 112. Data may be unencrypted for processing and presentation. A secure connection may be established during the movement of data between the back end modules (106, 110) and the front end modules (104, 112).

In an embodiment, data in-transit may be configured to use a Transport Layer Security (TLS) protection to protect data in the PCS 200 and the HDPR+ system 800.

In an embodiment, the security module 116 may include an asset and risk registry to identify vulnerabilities, and a risk rating to help prioritize the mitigation.

In an embodiment, the security module 116 may be configured to calculate a likelihood rating for the likelihood of a vulnerability that may occur. Further, the security module 116 may be configured to calculate a consequence rating for the consequences based on the vulnerability.

In an embodiment, the likelihood rating may be marked as ‘Low’, ‘Likely’ and ‘Very Likely’ based on the likelihood of vulnerabilities.

In an embodiment, the likelihood rating may be marked as ‘Low’ for vulnerabilities that have a very low risk of occurring and have all mitigations in place.

In an embodiment, the likelihood rating may be marked as ‘Likely’ for vulnerabilities that are likely to occur and have all mitigations in place.

In an embodiment, the likelihood rating may be marked as ‘Very Likely’ for vulnerabilities that are very likely to occur, may have all mitigations in place, and there are major aspects of the vulnerability that cannot be controlled.

In an embodiment, the consequence rating may be categorized as ‘Low’, ‘Medium’ and ‘High’ based on the vulnerability.

In an embodiment, the consequence rating may be marked as ‘Low’ if the impact is highly localized and/or affecting few users, data is not compromised, some functionality is affected but work around is available, and solution may be put into place within hours.

In an embodiment, the consequence rating may be marked as ‘Medium’ if a large number of users are affected, impact is widespread, data is not compromised, some functionality is available but has no work around, some important functionality is still available to most users, and solution will take less than four hours.

In an embodiment, the consequence rating may be marked as ‘High’ if a significant number of users are affected, impact is widespread, data is compromised, security is compromised for a significant number of patients, significant amounts of patient data may be inaccessible, and solution will take greater than or equal to four hours.

In an embodiment, a risk rating associated with potential vulnerabilities may be determined as the product of the likelihood rating and the consequence rating. This ranking may be used to determine the priority of effort to mitigate the risks.

In an embodiment, the final risk rating may assume that all mitigation actions have been implemented. Further, the final risk rating may assume that all system components have completed their multiple stages of testing, from unit testing to user acceptance testing. The final stage may be the authorization to release the system into production.

In an embodiment, the risk rating may be marked as ‘Low’ for the following possibilities: a) the likelihood rating is Low and the consequence rating is Low, b) the likelihood rating is Low and the consequence rating is Medium, and c) the likelihood rating is Likely and the consequence rating is Low.

In an embodiment, the risk rating may be marked as ‘Medium’ for the following possibilities: a) the likelihood rating is Low and the consequence rating is High, b) the likelihood rating is Likely and the consequence rating is Medium, and c) the likelihood rating is Very Likely and the consequence rating is Low.

In an embodiment, the risk rating may be marked as ‘High’ for the following possibilities: a) the likelihood rating is Likely and the consequence rating is High, b) the likelihood rating is Very Likely and the consequence rating is Medium, and c) the likelihood rating is Very Likely and the consequence is High.

In an embodiment, the security module 116 may be configured to put mitigation in place for asset types such as, but not limited to, Patient ID and Password, Clinician ID and Password and Patient Healthcare Number, Patient Internal ID, Administrator and Tech Support ID's and Passwords, Blockchain Public and Private Keys, Patient Data in Graph DB, Graph DB Configuration, Application Code, Front-End API, Back-End API, Blockchain API, External Data Source Interoperability, Blockchain Nodes, Front-End and Back-End server capabilities, and Network.

In an embodiment, the security module 116 may be configured to assign a likelihood rating, a consequence rating and a risk rating to both system and the patient 102 based on the vulnerability, and the possible threat(s) associated with the asset type. The security module 116 may further be configured to put a mitigation in place to mitigate the perceived threat(s).

In an embodiment, the Patient ID and Password may be vulnerable, as a user's credentials may be compromised in multiple ways. The security module 116 may assign a likelihood rating of ‘Likely’ to both system 100 and the patient 102, as it is likely that credentials of many users 102 will be compromised.

In an embodiment, the asset type Patient ID and Password may need mitigation against the perceived threats, wherein the perceived threats may be, a malicious person may sign in as the patient, ID's may be easily obtained and Password's (PSW's) may be determined. The security module 116 may assign the consequence rating of ‘Low’ for the system 100 as the seriousness of the threat to the system 100 is minor when compared to the patient 102. A consequence rating of ‘High’ may be assigned for the patient 102 as the patient data is exposed and privacy decisions may be changed.

In an embodiment, the security module 116 may be configured to calculate the risk rating based on the likelihood rating and the consequence rating. In this case, the security module 116 may assign a risk rating of ‘Low’ for the system 100 and ‘High’ for the patient 102 as there is little control over the patient's adherence to good security behavior.

In an embodiment, the security module 116 may be configured to have mitigations in place to reduce risk against the threats associated with the asset type Patient ID and PSW such as, but not limited to, limit number of login attempts, two factor authentication, force answers to additional questions to login, require name, password and healthcare ID (e.g., OHIP #), display date of last login during login, limit time without any activity, notification of use of ID and the type of use (view and change), view log of anyone who viewed their data and when (including themselves), and check that patient is not being emulated by a machine.

In an embodiment, the Clinician ID, Password and the Patient Healthcare number may be vulnerable as the clinician 114 may leave a terminal unattended and the clinician credentials may be compromised. The security module 116 may assign a likelihood rating of ‘Likely’ to both the system 100 and the patient 102, as it is likely that some credentials may be compromised, or computers may be left unattended.

In an embodiment, the asset type Clinician ID, Password and the Patient Healthcare number may need mitigation against the perceived threats, wherein the perceived threats may be, a malicious person signing in as the patient, IDs may be easily obtained, PSW's may be determined, and the clinicians 114 may leave the season unattended. The security module 116 may assign a consequence rating of ‘Low’ for the system 100 as the seriousness of the threat to the system may be minor when compared to the patient. A consequence rating of ‘High’ may be assigned for the patient 102 as the patient data of all the registered users may be exposed, and unauthorized access to the terminal may allow the unauthorised user to view patient data as well as declare an emergency.

In an embodiment, the security module 116 may be configured to calculate and assign a risk rating of ‘Low’ for the system 100 and ‘High’ for the patient 102 as there may be little control over the patient's/clinician's adherence to good security behavior.

In an embodiment, the security module 116 may be configured to have mitigations in place to reduce risk against the threats associated with the asset type Clinician ID, Password and Patient Healthcare Number such as, but not limited to, Limit number of login attempts, two factor authentication, force answers to additional questions, notification of use of ID, display date of last login, limit time without any activity, log viewing of patient data, log declaration of an emergency so as to view all the patient's data, limit the number of patients viewed per day, expand the additional patient ID information required such as, the patient's name (may allow registered emergency room doctors to read with minimal additional patient ID), check that clinician is not being emulated by a machine, and front-End calls Back-End via API for authentication.

In an embodiment, the asset type Patient Internal ID may be a unique internal ID associated with each patient's healthcare ID. The unique internal ID may be a key indexed to the stored patient data and may only be used by the Back-End service. The Patient Internal ID may be vulnerable, as the credentials to accounts with access to back-end may be compromised or used in an unauthorised manner. The security module 116 may assign a likelihood rating of ‘Low’ to system 100, as with mitigation in place it will be very difficult to obtain this information.

In an embodiment, the asset type Patient internal ID may need mitigation against the perceived threat, wherein the perceived threat may be, edit access to the back-end service and internal ID table may allow changing a user's internal ID. The security module 116 may assign a consequence rating of ‘High’ for the system as the this would be a serious breach with broad consequences, and the hacker may change their internal ID to view any patient's data.

In an embodiment, the security module 116 may calculate and assign a risk rating of ‘Medium’ to the system as the severity is extreme and must have full mitigation in place.

In an embodiment, the security module 116 may be configured to have mitigations in place to reduce risk against the threats associated with the asset type Patient internal ID such as, but not limited to, encrypt the table containing the internal ID mapping to the patient ID, encrypt the internal ID on the blockchain transactions, and allow only the Back-End service to access the mapping table and decrypt the internal ID.

In an embodiment, the Administrator and Tech Support IDs and Passwords may be vulnerable as the credentials to accounts with access to back-end may be compromised or used in an unauthorized manner. The security module 116 may assign a likelihood rating of ‘Low’ to the system 100, as the administrators may be sloppy.

In an embodiment, the asset type Administrator and Tech Support IDs and Passwords may need mitigation against the perceived threats, wherein the perceived threats may be, administrators may have access and the authority to perform tasks that may inhibit the proper operation of the system, and possible read access to external EHRDB's 816 and laboratory databases. The security module 116 may assign a consequence rating of ‘High’ for the system 100 as the proper operation of the system is compromised and the patient data is exposed.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associated with the asset type Administrator and Tech Support IDs and Passwords such as, but not limited to, use role-based authorization to limit access to the system, use IDs that are assigned to specific individuals rather than shared IDs, immediately alter access rights and passwords if the role of a user changes, administrative tasks should be designed to restrict the administrator to what commands they may execute and not how they execute, monitor and record the activities of staff, enact strong processes, controls and training, two factor authentication for all server access, restrict high risk changes to development systems and promote to production through a strong QA process, front-End calls Back-End via API for authentication, log all activities, and force regular password changes.

In an embodiment, the asset type Blockchain Public and Private Keys may be vulnerable as there may be an unauthorized access to blockchain keys. The security module 116 may assign a likelihood rating of ‘Low’ to the system 100, as keys may be used in automated processes and are well protected.

In an embodiment, the asset type Blockchain Public and Private Keys may need mitigation against the perceived threats, wherein the perceived threats may be, access to the keys which may be used to run smart contracts to view and execute transactions. The security module 116 may assign a consequence rating of ‘High’ for the system 100 as the patient data may be exposed, transactions may be executed on the blockchain, and if the keys are changed then the operation of the system would be compromised.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associated with the asset type Blockchain Public and Private Keys such as, but not limited to, blockchain keys should be accessible only to the Back-End, keys should be encrypted and only an authorized account can encrypt or decrypt the data, patient data on the blockchain should be encrypted, secure the blockchain API so only an authorized account can access the API, use a permissioned blockchain, and the blockchain contains only privacy data (no patient data is referenced or stored on the blockchain).

In an embodiment, the asset type Patient Data in Graph DB 108 may be vulnerable as unauthorized access or use of graph DB gives access to unencrypted patient data. The security module 116 may assign a likelihood rating of ‘Low’ to the system 100 as the data administrators will have access and can be security sloppy.

In an embodiment, the asset type Patient Data in Graph DB 108 may need mitigation against the perceived threats wherein the perceived threats may be, the Neo4j Graph DB technology used to store patient data does not currently support native encryption of node properties (in which patient data is stored), and encrypting the data would require customization to the Neo4j code and may slow query performance. The security module 116 may assign a consequence rating of ‘High’ for the system 100 as this would be a serious breach with broad consequences, anyone with direct access to the graph DB 108 may read all patient data, and if the account used to access the graph DB had sufficient authority then the patient data may be altered or created.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associate with the asset type Patient Data in Graph DB 108 such as, but not limited to, allow only the Back-End service to have access to the graph DB 108, use the patient internal ID within the graph DB 108 to identify a specific patient, encrypt just the patient data that would easily allow determination of the identity of the patient (for example, demographic data and would limit the performance impact of encryption), store patient data in the graph DB 108 temporarily (the length of time it is kept in the graph DB 108 would be based on an algorithm that would take into account, time since the data was last used and age of the data), adhere to the data-at-rest, data-in-use and data-in-transit requirements, and point in time recovery if data is compromised.

In an embodiment, the asset type Graph DB Configuration may be vulnerable as there is a possibility of sloppy testing and requirements gathering. The security module 116 may assign a likelihood rating of ‘Low’ to the system 100 as sometimes processes are not followed, or work is sloppy.

In an embodiment, the asset type Graph DB Configuration may need mitigation against the perceived threats, wherein the perceived threats may be, unauthorized or poorly considered updates to the graph DB 108 node and relationship configuration. The security module 116 may assign a consequence rating of ‘High’ to the system 100 as this may affect the treatment given to the patients 102, and alteration of nodes and relationships would compromise the operation of the system and result in the wrong or false data being displayed.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associated with the asset type Graph DB Configuration such as, but not limited to, allow only the Back-End service to have update and load access to the graph DB 108, remove update access in production and enable on a temporary basis only if required, all configuration additions and changes should be approved by a committee of clinicians tasked with maintaining the integrity and correctness of the configuration, and strong QA process.

In an embodiment, the asset type Application code may be vulnerable as there may be sloppy testing, requirement gathering and code. The security module 116 may assign a likelihood rating of ‘Low’ for the system 100 as the coders, analysts, and other staff may make mistakes.

In an embodiment, the asset type Application code may need mitigation against the perceived threats, wherein the perceived threats may be, the quality of the system code in terms of integrity, correctness, availability, scalability may be compromised. The security module 116 may assign a consequence rating of ‘High’ for the system 100 as the healthcare provided to the patients 102 and their trust in clinicians 114 will be lessened and potentially harmful to the health of the patients 102 if the application does not perform as required, availability, security and stability of the application is compromised, and patient data may be exposed including data flagged as private.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associate with the asset type Application code such as, but not limited to, implement strong development and QA processes such as the use of Docker to sandbox applications, ensure code is secured from unauthorized changes, ensure strong backup and restore processes are in place, provide for real-time fail-over, use audit logs, security software in place to scan all code changes for vulnerabilities, and compartmentalize code to reduce the complexity of testing.

In an embodiment, the Front-End API, which is the entry point for all requests for the system, may be vulnerable as a system that provides private healthcare information may be an enticing target for attacks. The security module 116 may assign a likelihood rating of ‘Likely’ for the system, as a healthcare system would be an inviting target for hackers.

In an embodiment, the asset type Front-End API may need mitigation against the perceived threats, wherein the perceived threats may be, denial of service attacks, and attempts to access front-end services. The security module 116 may assign a consequence rating of ‘High’ for the system 100 as the denial of service attacks may prevent the system from servicing legitimate requests and patient's data may be exposed.

In an embodiment, the security module 116 may be configured to calculate and assign a risk rating of ‘High’ for the system 100 as the severity is extreme and must have full mitigation in place.

In an embodiment, the security module 116 may be configured to have mitigations in place to reduce risk against the threats associate with the asset type Front-End API such as, but not limited to, configure the API to accept only authorized systems and registered users coming from authorized web servers, encrypt traffic from the public network (HTTPS), implement Denial of Service defences, and protect against injection attacks.

In an embodiment, the asset type Back-End API, wherein the Back-End API is an entry point for all system level requests to the Back-End, may be vulnerable as a system that provides private healthcare information may be an enticing target for attacks. The security module 116 may assign a likelihood rating of ‘Likely’ to the system 100 as a breach of the front-end can lead to the ability to submit transaction to the back-end.

In an embodiment, the asset type Back-End API may need mitigation against the perceived threats, wherein the perceived threats may be, denial of service attacks, and attempts to access back-end services. The security module 116 may assign a consequence rating of ‘High’ for the system 100 as unauthorized access patient data may result in patient data being exposed, and denial of service attacks would prevent the system from servicing legitimate requests.

In an embodiment, the security module 116 may be configured to calculate and assign a risk rating of ‘High’ for the system 100 as the severity is extreme and must have full mitigation in place.

In an embodiment, the security module 116 may be configured to have mitigations in place to reduce risk against the threats associate with the asset type Back-End API such as, but not limited to, place the Back-End on a subnet, only accept requests from the Front-End, and use strong authentication and authorization such as Double Data Encryption Standard (2DES) or Advanced Encryption Standard (AES) encryption.

In an embodiment, the asset type Blockchain API (for system requests to the blockchain) may be vulnerable as knowing a patient's 102 privacy decisions (on the blockchain) may indicate to an attacker that the patient 102 has something to hide. The security module 116 may assign a likelihood rating of ‘Likely’ for system as a blockchain with patient privacy decisions may be enticing to a hacker.

In an embodiment, the asset type Blockchain API may need mitigation against the perceived threats, wherein the perceived threats may be, unauthorized requests to Blockchain API, and denial of service attack. The security module 116 may assign a consequence rating of ‘High’ for the system 100 as denial of service attacks may prevent the system from servicing legitimate requests, and unauthorized access may result in patient privacy decisions being exposed.

In an embodiment, the security module 116 may be configured to calculate and assign a risk rating of ‘High’ for the system 100 as the severity is extreme and must have full mitigation in place.

In an embodiment, the security module 116 may be configured to have mitigations in place to reduce risk against the threats associate with the asset type Blockchain API such as, but not limited to, only accept requests from the Back-End, use strong authentication and authorization such as 2DES or AES encryption, and run blockchain in a subnet different from the Front-End accepting requests from only the Back-End system.

In an embodiment, the asset type External Data Source Interoperability may be vulnerable as the source EHRDB's 816 and laboratory databases are external and their operation is the responsibility of third parties. The security module 116 may assign a likelihood rating of ‘Likely’ to the system 100 as outages may happen to external services that are beyond our control.

In an embodiment, the asset type External Data Source Interoperability may need mitigation against the perceived threats, wherein the perceived threats may be, receipt of false data from an external data source, and patient data not available due to source system being inaccessible. The security module 116 may assign a consequence rating of ‘Medium’ for the system 100 as the external data if inaccurate may affect the patient's 102 healthcare, and unavailable data will make clinicians less efficient.

In an embodiment, the security module 116 may be configured to calculate and assign a risk rating of ‘High’ for the system 100 as the severity is extreme and must have full mitigation in place.

In an embodiment, the security module 116 may be configured to have mitigations in place to reduce risk against the threats associated with the asset type External Data Source Interoperability such as, but not limited to, the connection between the system and external suppliers of patient data should have strong encryption and authentication, and the standards recommended by the government should be analysed.

In an embodiment, the asset type Blockchain Nodes may be vulnerable as knowing the patient's privacy decisions (on the blockchain) may indicate to an attacker the patient 102 has something to hide. The security module 116 may assign a likelihood rating of ‘Low’ to the system as mitigation makes this unlikely.

In and embodiment, the asset type Blockchain Nodes may need mitigation against perceived threat, wherein the perceived threat may be, numerous types of attacks to which the blockchain nodes are susceptible. The security module 116 may assign a consequence rating of ‘High’ for the system 100 as an attack on a blockchain may result in the blockchain being inoperable or exposes patient privacy decision, and may lead to an exposure of patient's 102 private data.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associate with the asset type Blockchain Nodes such as, but not limited to, all nodes must authenticate themselves and a trust mechanism should be implemented to decide if the node is trustworthy and only then the node will be allowed to join the network, and an alternative would be to restrict the nodes to one entity such as in government data centres.

In an embodiment, the asset type Front-End and Back-End server capabilities may be vulnerable as unanticipated service loads may stress systems, and may be due to platform or data centre outages. The security module 116 may assign a likelihood rating of ‘Low’ to the system as due to enough industry experience, most issues are known and may be prepared for.

In an embodiment, the asset type Front-End and Back-End server capabilities may need mitigation against the perceived threat, wherein the perceived threat may be, anticipated events. Anticipated threats may be prepared for, but unanticipated events may create problems that will slow resolution of the issues. The security module 116 may assign a consequence rating of ‘High’ for the system 100 as there may be widespread impact and affect healthcare services to large numbers of patients, and poor service availability, stability, scalability and performance may compromise the healthcare provided to patients and their trust in the healthcare system.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associate with the asset type Front-End and Back-End server capabilities such as, but not limited to, the servers must be properly specified to meet the requirements, this would include consideration for security, fail-over, scalability, performance and stability, and replication of data and services.

In an embodiment, the asset type Network may be vulnerable as networks tie all the components of a system together and represent a weak point in any design. The security module 116 may assign a likelihood rating of ‘Low’ for system 100 as it is unlikely for core services but, likely may occur at the services used at the receiving end (as an example: first responders requirement for wireless service or doctors' offices internet connections).

In an embodiment, the asset type Network may need mitigation against the perceived threat, wherein the perceived threat may be, unavailability of patient data and services to patients, clinicians and first responders. The security module 116 may assign a consequence rating of ‘High’ for the system 100 as patient healthcare may be negatively impacted in serious scenarios, and delays in receiving healthcare.

In an embodiment, the security module 116 may be configured to calculate and assign a risk rating of ‘Medium’ for the system 100 as the severity is extreme and must have full mitigation in place.

In an embodiment, the security module 116 may be configured to have mitigations in place to reduce risk against the threats associated with the asset type Network such as, but not limited to, anti-virus and anti-malware services, firewalls, monitor traffic in and out through the firewalls, place public facing services and external IP's on a separate network segment or DMZ, server access logs, monitor threat activity, data loss prevention, server hardening (remove unnecessary services and applications), keep software up-to-date and patched, password management, security policies and management, defence against phishing and spoofing messages, user account management, security threat training and penetration tests, 7/24 Security Operations Centre, and utilize multiple different network services.

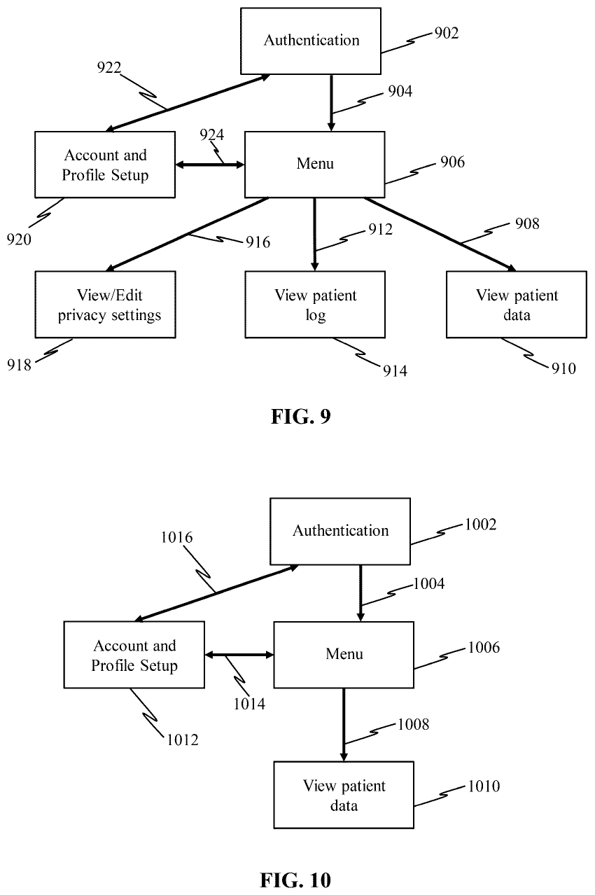

FIG. 9 illustrates the user interface displayed for the patient 102 on a display device corresponding to the patient 102, in accordance with an embodiment.

In an embodiment, the patient 102 may be a new user. By choosing the ‘New account’ option 922, the patient may be allowed to access the account and profile setup 920, wherein the patient 102 may enter data for new account and profile creation. After saving the changes, the system 100 may direct the patient back to the Authentication 902.

In an embodiment, the patient 102 may need to be a patient registered user of the healthcare system comprising the healthcare data access system 100. As an example, in Ontario, a patient 102 would not be able to configure an account unless they have a valid OHIP card. Some profile settings may not be allowed to be changed if they differ from the patients OHIP profile.

In an embodiment, an administrator may create a Patient node (P) in the graph DB 108 upon registration to the healthcare system by the patient 102.

In an embodiment, the patient 102 may be an existing user and request to edit the profile settings 922. The patient 102, after updating the profile information 920, may be directed 924 to the Menu 906 after clicking ‘Next’.

In an embodiment, the system 100 may verify the credentials of the patient 102 through authentication 902. After login 904 the patient 102 may view the Menu 906. At Menu 906, the patient 102 may choose ‘View healthcare data’ option 908 to view patient data 910. The patient 102 may choose ‘View log’ option 912 to view patient log 914. Further, the patient may choose ‘Privacy settings’ option 916, wherein the ‘Privacy settings’ option 916 allows the patient 102 to View/Edit privacy settings 918.

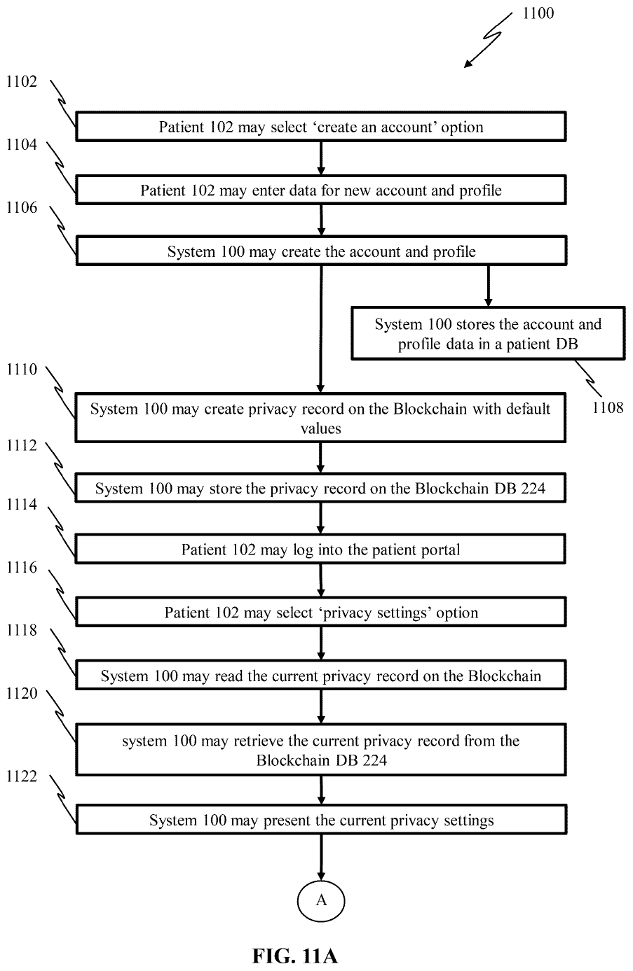

FIG. 10 illustrates the user interface displayed for the clinician 114 on a display device corresponding to the clinician 114, in accordance with an embodiment.

In an embodiment, the clinician 114 may be a new user. By choosing the ‘New account’ option 1016, the clinician 114 may be allowed to access the account and profile setup 1012, wherein the clinician 114 may enter data for new account and profile creation. After saving the changes, the system 100 may direct the clinician 114 back to the Authentication 1002.

In an embodiment, the clinician 114 may need to be a registered user of the healthcare system comprising the healthcare data access system 100 of the practising jurisdiction(s).

In an embodiment, the clinician 114 may be an existing user and request to edit the profile settings 1012. The clinician 114, after updating the profile information, may be directed 1014 to the Menu 1006 after clicking ‘Next’.

In an embodiment, the clinician 114 may not be allowed to edit all data related to the account and the profile information of the clinician 114.

In an embodiment, the system 100 may verify the credentials of the clinician 114 through authentication 1002. After login 1004 the clinician 114 may view the Menu 1006. At Menu 1006, the clinician 114 may choose ‘View patient data’ option 1008 to view patient data 1010.

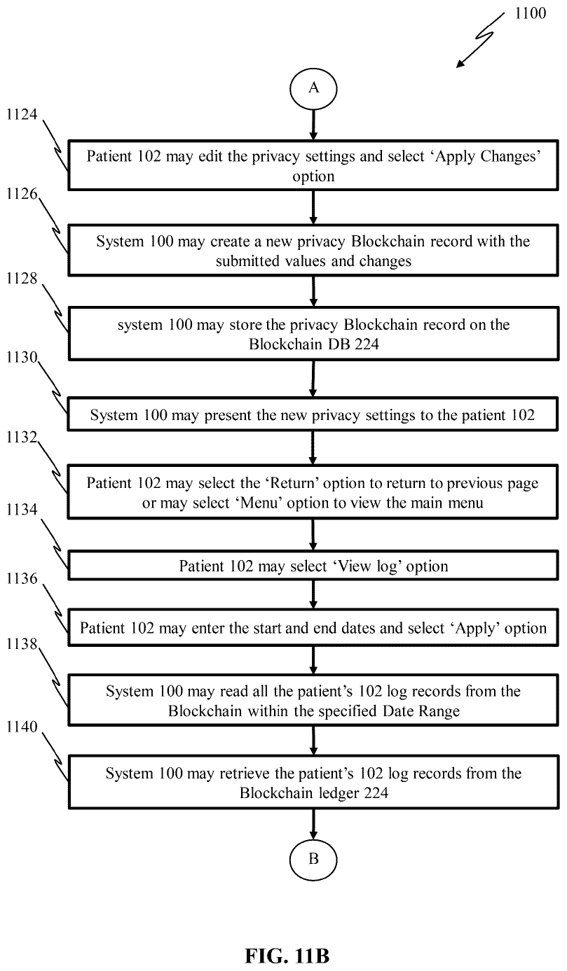

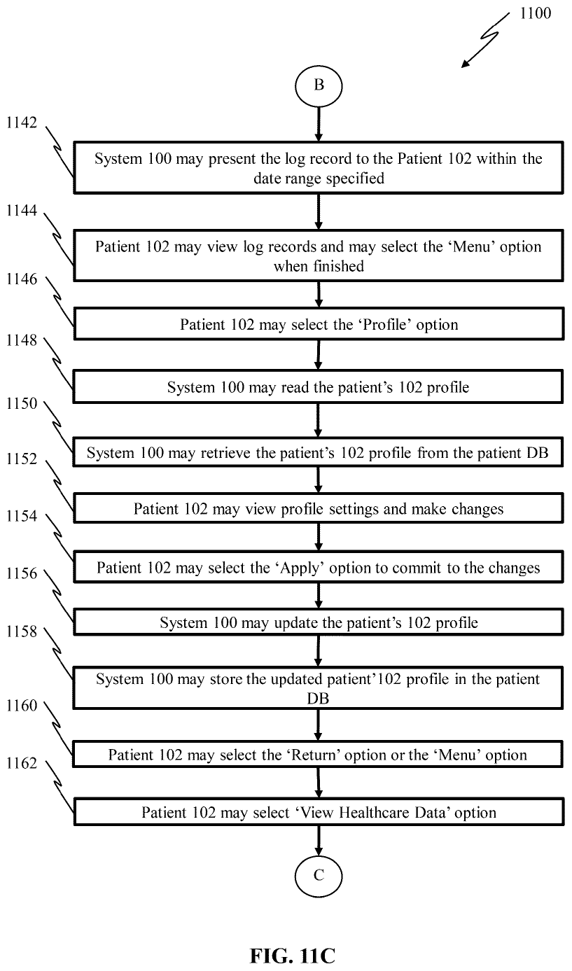

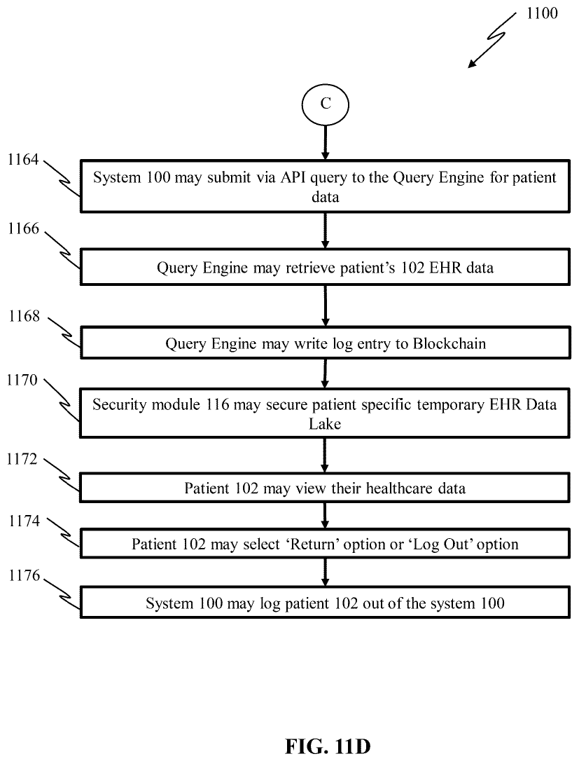

FIG. 11A-11D illustrate a flow chart depicting the workflow of the patient 102 using the PCS 200 within the system 100, in accordance with an embodiment. Referring FIG. 11A, at step 1102, the patient 102 may choose ‘Create an Account’ option.

At step 1104, the patient 102 may enter data for new account and profile creation.

At step 1106, the system 100 may create the account and profile for the patient 102.

At step 1108, the system 100 may store the account and profile data in a patient database (patient DB), wherein the patient DB may be the graph DB 108.

At step 1110, the system 100 may create privacy record on the Blockchain with default values.

At step 1112, the system 100 may store the privacy record on the Blockchain DB 224.

At step 1114, the patient 102 may log into the patient portal.

At step 1116, the patient 102 may select ‘Privacy Settings’ option.

At step 1118, the system 100 may read the current privacy record on the Blockchain.

At step 1120, the system 100 may retrieve the current privacy record from the Blockchain DB 224.

At step 1122, the system 100 may present the current privacy settings to the patient 102.

At step 1124, the patient 102 may edit the privacy settings and selects ‘Apply Changes’ option. Here, privacy settings may be edited based on the privacy categories and the date ranges of the healthcare data.

At step 1126, the system 100 may create a new privacy Blockchain record with the submitted values and changes.

At step 1128, the system 100 may store the privacy Blockchain record on the Blockchain DB 224.

At step 1130, the system 100 may present the new privacy settings to the patient 102.

At step 1132, the patient 102 may select the ‘Return’ option to return to previous page or may select ‘Menu’ option to view the main menu.

At step 1134, the patient 102 may select the ‘View Log’ option.

At step 1136, the patient 102 may enter the start and end dates and selects ‘Apply’ option.

At step 1138, the system 100 may read all the patient's 102 log records from the Blockchain within the specified Date Range.

At step 1140, the system 100 may retrieve the patient's 102 log records from the Blockchain ledger 224.

At step 1142, the system 100 may present the log records to the patient 102 within the date range specified.

At step 1144, the patient 102 may view log records and may select the ‘Menu’ option when finished.

At step 1146, the patient 102 may select the ‘Profile’ option.

At step 1148, the system 100 may read the patient's 102 profile.

At step 1150, the system 100 may retrieve the patient's 102 profile from the patient DB.

At step 1152, the patient 102 may view profile settings and make changes.

At step 1154, patient 102 may select the ‘Apply’ option to commit to the changes.

At step 1156, the system 100 may update the patient's 102 profile.

At step 1158, the system 100 may store the updated patient' 102 profile in the patient DB.

At step 1160, the patient 102 may select the ‘Return’ option or the ‘Menu’ option.

At step 1162, the patient 102 may select ‘View Healthcare Data’ option.

At step 1164, the system 100 may submit via API query to a Query Engine for patient data.

At step 1166, the Query Engine may retrieve patient's 102 EHR data.

At step 1168, the Query Engine may write log entry to the Blockchain.

At step 1170, the security module 116 may secure patient specific temporary EHR Data Lake.

At step 1172, the patient 102 may view their healthcare data.

In an embodiment, the patient 102 viewing their healthcare data will be displayed with that data's privacy categorization. This may make it easier for the patient to make privacy decisions. Further, the patient may simply click on the data they are viewing and assign a privacy decision to that data without having to go to the edit screen.

At step 1174, the patient 102 may select ‘Return’ option or log Out′ option.

At step 1176, the system 100 may log patient 102 out of the system 100.

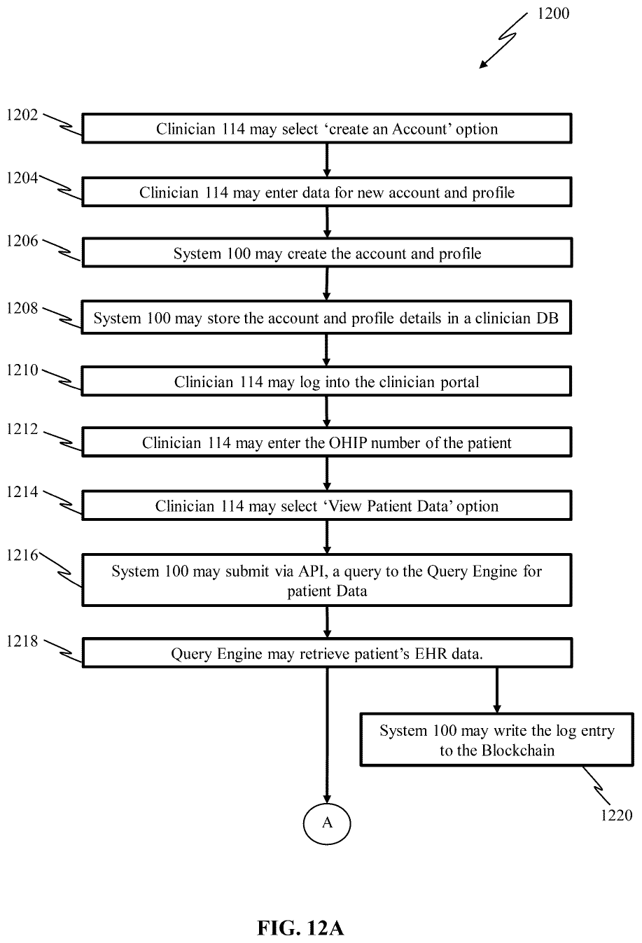

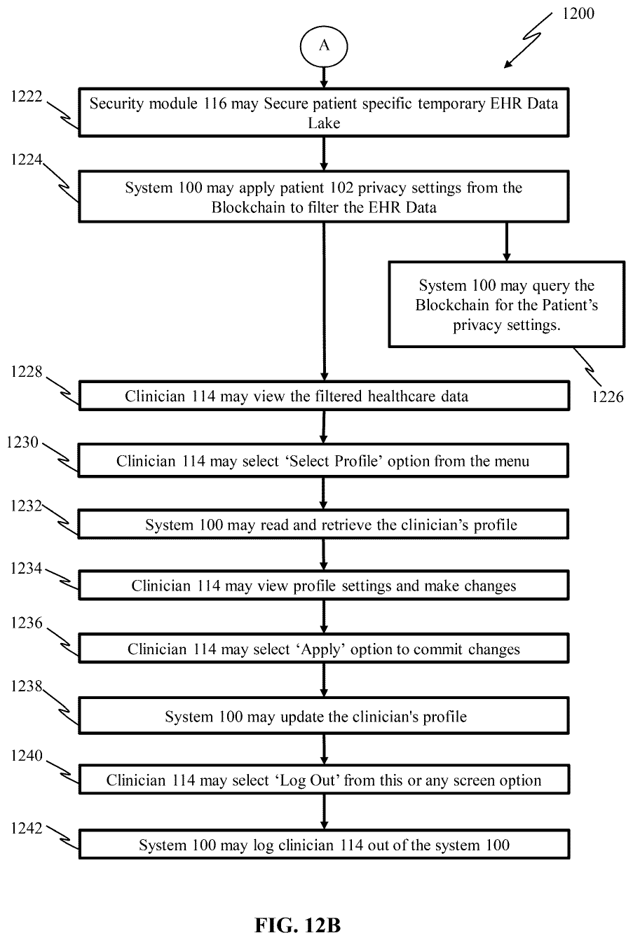

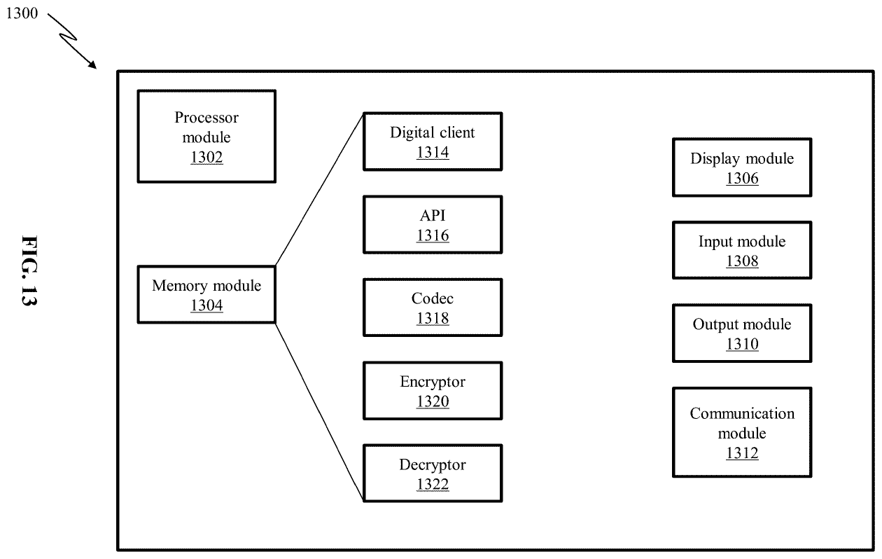

FIG. 12A-12B illustrate a flow chart depicting the workflow of the clinician 114 using the HDPR+ system 800, in accordance with an embodiment. Referring FIG. 12, at step 1202, the clinician 114 may select ‘Create an Account’ option.

In an embodiment, the clinician 114 may be assigned a user ID and a default password as part of an administrative process. The clinician 114 may be able to edit a defined part of their profile.

At step 1204, the clinician 114 may enter data for new account and profile.

At step 1206, the system 100 may create the account and profile for the clinician 114.

At step 1208, the system 100 may store the account and profile details in a clinician DB. In an embodiment, the clinician DB may be a separate database administering clinician accounts.

In an embodiment, a clinician may also be a patient. The system 100 may be configured to assign a separate patient account for the clinician who is a patient.

At step 1210, the clinician 114 may log into the clinician portal.

At step 1212, the clinician 114 may enter the OHIP number of the patient.

At step 1214, the clinician 114 may select ‘View Patient Data’ option.

In an embodiment, the clinician 114 may select a default Schema assigned to them to view patient data.

At step 1216, the system 100 may submit via API, a query to the Query Engine for patient data.

At step 1218, the Query Engine may retrieve patient's EHR data.

At step 1220, the system 100 may write the log entry to the blockchain.

At step 1222, the security module 116 may secure patient specific temporary EHR Data Lake.

At step 1224, the system 100 may apply patient 102 privacy settings from blockchain to filter the EHR data.

At step 1226, the system 100 may query the Blockchain for the patient's 102 privacy settings. The system 100 may be configured to check if emergency override is applicable in the current scenario.

At step 1228, the clinician 114 may view the patient's filtered healthcare data.

In an embodiment, the system 100 may be configured to ignore the patient's 102 privacy settings and present all the patient's healthcare data to the requesting clinician 114, when an emergency override is applicable.

At step 1230, the clinician 114 may select ‘Select Profile’ option from the menu.

At step 1232, the system 100 may read and retrieve the clinician's 102 profile.

At step 1234, the clinician 114 may view profile settings and make changes.

At step 1236, the clinician 114 may select ‘Apply’ option to commit changes.

At step 1238, the system 100 may update the clinician's 102 profile.

At step 1240, the clinician 114 may select log Out′ from this or any screen option.

At step 1242, the system 100 may log clinician 114 out of the system 100.

In an embodiment, the system 100 may be configured to attach a rating of accuracy of the data presented. The rating may comprise of five levels namely, Level 5, Level 4, Level 3, Level 2, and Level 1, representing a level of confidence of the accuracy of the data.

In an embodiment, Level 5 may be assigned when no reason is found to suspect the accuracy of the value.

In an embodiment, Level 4 may be assigned when the value was recorded by a human rather than a device.

In an embodiment, Level 3 may be assigned when the value is an anomaly. Statistical analysis computes a low level of confidence as compared to the values recorded at different times.

In an embodiment, Level 2 may be assigned when the value is outside the range it should be when compared to other related values.

In an embodiment, Level 1 may be assigned when the system from which the data is sourced has suspect accuracy as rated by other clinicians. Other mitigating factors are considered. As an example, the source of the data is from specific countries with less advanced healthcare systems.

In an embodiment, the pattern matching process may be utilized for use cases beyond checking for medical conditions. For example, to determine data accuracy for level 2 and above, and/or to estimate the impact of specific drugs on a patient.

In an embodiment, the privacy categorizations of the patient data may be customized to fit the requirements of healthcare systems in many jurisdictions with different cultures and regulations.

In an embodiment, the system 100 may be configured to provide multiple language support.

In an embodiment, the system 100 may be configured to support different data format standards.

In an embodiment, the system 100 may allow patients to simple view and click on the data they want flagged as private.

In an embodiment, the system 100 may allow the patients to click on data they wish to make available to all clinicians.

In an embodiment, the system 100 comprises of a memory device which may include graph DB 108, the schema DB 806 and the blockchain DB 224.

In an embodiment, plurality of computing devices and the system 100 may be configured to communicate over a network.

In an embodiment, a computing device 1400 may be configured to access the healthcare data access system 100.

In an embodiment, a first computing device of the patient 102 may be allowed to access the healthcare data access system 100 over the network.

In an embodiment, a second computing device of the clinician 114 may be allowed to access the healthcare data access system 100 over the network.

FIG. 13 illustrates the computing device 1300, in accordance with an embodiment.

In an embodiment, the computing device 1300 may comprise a processor module 1302, a memory module 1304, a display module 1306, input modules 1308, output modules 1310 and a communication module 1312.

The processor module 1302 may be implemented in the form of one or more processors and may be implemented as appropriate in hardware, computer-executable instructions, firmware, or combinations thereof. Computer-executable instruction or firmware implementations of the processor module 1302 may include computer-executable or machine-executable instructions written in any suitable programming language to perform the various functions described.

The memory module 1304 may include a permanent memory such as hard disk drive, may be configured to store data, and executable program instructions that are implemented by the processor module. The memory module 1304 may be implemented in the form of a primary and a secondary memory. The memory module 1304 may store additional data and program instructions that are loadable and executable on the processor module 1302, as well as data generated during the execution of these programs. Further, the memory module 1304 may be volatile memory, such as random-access memory and/or a disk drive, or non-volatile memory. The memory module 1304 may comprise of removable memory such as a Compact Flash card, Memory Stick, Smart Media, Multimedia Card, Secure Digital memory, or any other memory storage that exists currently or may exist in the future.

In an embodiment, the memory module 1304 may further comprise a digital client 1314, an API 1316, a codec 1318, an encryptor 1320 and a decryptor 1322. The digital client 1314 may be a web browser or a software application enabling multiple screen sharing simultaneously, wherein the digital client 1314 may further comprise a first digital client display interface. The codec 1318 may include computer-executable or machine-executable instructions written in any suitable programming language to perform compress outgoing data and decompress incoming data. The encryptor 1320 may encrypt the data being sent and decryptor 1322 may decrypt the incoming data.

The display module 1306 may display an image, a video, or data to a user. For example, the display module 1306 may include a panel, and the panel may be an LCD, LED or an AM-OLED. The display module 1306 may be configured to display the patient UI and the clinician UI.

The input modules 1308 may provide an interface for input devices such as keypad, touch screen, mouse and stylus among other input devices. In an embodiment, the input modules 1308 includes a camera and a microphone.

The output modules 1310 may provide an interface for output devices such as display screen, speakers, printer and haptic feedback devices, among other output devices.

The communication module 1312 may be used by the computing device 1300 to communicate with the system 100 over the network. The communication module 1312, as an example, may be a GPRS module, or other modules that enable wireless communication.

In an embodiment, a processor 118 may be implemented in the form of one or more processors and may be implemented as appropriate in hardware, computer-executable instructions, firmware, or combinations thereof. Computer-executable instruction or firmware implementations of the processor 118 may include computer-executable or machine-executable instructions written in any suitable programming language to perform the various functions described.

The claimed invention provides a plenty of advantages. The system 100 allows the patients to have more say in the privacy of their data and improved transparency into how their data is being used. The system 100 also makes it easier for the average patient to make privacy decisions on their healthcare data without requiring a strong knowledge of medical terminology. Further, by allowing the patient to make data ranges private, it is easier to handle healthcare events that may require multiple related pieces of healthcare data to be flagged as private.

Other notable advantage to the system 100 is the combination of capabilities to improve the management that patients have over their healthcare data privacy. This includes the ability for a patient to view a log of who viewed their data and when it was requested. It provides the ability of the patient to view their own healthcare data. This improves the ability of the patient to identify data they want to make private, plus the ability to pre-authorize access to their data not flagged as private. This can speed the clinician's access to the patient's data and reduces the effort required of the patient and clinician. Using a single system to handle privacy improves the accuracy and consistency of the patient's privacy decisions, as compared to an environment where the patient must interface with multiple different security systems. Additionally, the graph DB 108 of the proposed system 100 is configured to delete data which is unused for a period of time, thus increasing the efficiency of the overall system by having significantly less data to process through. Further, the proposed system provides significant time savings to the clinicians by allowing them to choose from pre-defined categories which further allows them to treat more patients in a day.

The processes described above is described as a sequence of steps, this was done solely for the sake of illustration. Accordingly, it is contemplated that some steps may be added, some steps may be omitted, the order of the steps may be re-arranged, or some steps may be performed simultaneously.

The example embodiments described herein may be implemented in an operating environment comprising software installed on a computer, in hardware, or in a combination of software and hardware.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

1. A healthcare data access system for improving healthcare data usability for clinicians and patients comprising:
at least one computer readable memory device comprising healthcare data of the patients; and
at least one processor, wherein the processor is configured to:
receive from a first computing device of a patient, privacy decisions to be applied to the patient's healthcare data;
receive from a second computing device of a clinician a request for the healthcare data of the patient, wherein the request comprises a selection of a schema, wherein the schema is selected from a plurality of schemas, wherein each of the schemas corresponds to a pre-defined search query;
retrieve a set of selected healthcare data from the healthcare data of the patient, based on the request comprising the selected schema; and
communicate:
a permissible healthcare data to the second computing device, wherein the permissible healthcare data from the selected healthcare data is obtained by filtering the selected healthcare data by applying the privacy decisions received from the first computing device; or
the set of selected healthcare data from the healthcare data of the patient, by overriding the privacy decisions received from the first computing device, when an emergency is declared by the second computing device.

2. The healthcare data access system as claimed in claim 1, wherein the processor is configured to present a plurality of privacy decision categories to the first computing device of the patient, wherein one or more of the privacy decision categories are received as selection by the patient via the first computing device to define the privacy decisions to be applied to the patient's healthcare data.

3. The healthcare data access system as claimed in claim 1, wherein the processor is configured to delete the healthcare data from the computer readable memory device when the said healthcare data is not accessed for a set period.

4. The healthcare data access system as claimed in claim 3, wherein the processor is further configured to:
retrieve at least a portion of the healthcare data, from an external memory device after receiving the selected schema from the second computing device of the clinician, which is not present in the computer readable memory device;
transform and store the retrieved healthcare data in the computer readable memory device;
retrieve the set of selected healthcare data from the computer readable memory device, based on the request comprising the selected schema;
perform pattern matching based on the set of selected healthcare data to identify possible medical conditions corresponding to the patient; and
identify medical references associated with the medical conditions and metadata associated with the set of selected healthcare data.

5. The healthcare data access system as claimed in claim 4, wherein the healthcare data of the patients is stored in the computer readable memory device in the form of plurality of nodes holding the healthcare data at a granular level, wherein the processor is configured to create plurality of relationships among the plurality of nodes.

6. The healthcare data access system as claimed in claim 5, wherein the processor is configured to delete unused plurality of nodes and with the relationships between the nodes after a set period.

7. The healthcare data access system as claimed in claim 5, wherein the processor is configured to perform the pattern matching on the healthcare data present in the plurality of nodes and with the relationships between the nodes.

8. The healthcare data access system as claimed in claim 5, wherein the computer readable memory device allows access to an entity node by:
a top-down query, wherein the top-down query starts with a schema node;
a bottom-up query, wherein the bottom-up query starts with a patient node.

9. The healthcare data access system as claimed in claim 1, wherein the pre-defined search query associated with the schema is customisable by the clinician.

10. The healthcare data access system as claimed in claim 1, wherein the schema comprises instructions on how the healthcare data should be presented on the second computing device, wherein the processor is configured to communicate the healthcare data, to the second computing device, to be presented in compliance with the instructions in the schema.

11. The healthcare data access system as claimed in claim 10, wherein the system attaches a rating of accuracy of the data presented to the clinician, wherein the rating of accuracy represents a level of confidence of the accuracy of the data presented to the clinician.

12. The healthcare data access system as claimed in claim 1, wherein the system comprises of a security module, wherein the security module is configured to generate a likelihood rating based on the likelihood of a vulnerability.

13. The healthcare data access system as claimed in claim 12, wherein the security module is further configured to generate a consequence rating based on the consequences of the vulnerability.

14. The healthcare data access system as claimed in claim 13, wherein the security module is configured to calculate a risk rating, wherein the risk rating is a product of the likelihood rating and the consequence rating.

15. The healthcare data access system as claimed in claim 1, wherein the computer readable memory device comprises a blockchain database, wherein the blockchain database is configured to:
store the privacy decisions applied to the patient's healthcare data by the patient; and
store plurality of activity log entries.
